(19) United States
(12) Patent Application Publication
(10) Pub. No.: US 2023/0025725 A1
(43) Pub. Date: Jan. 26, 2023

(54) STORAGE MEDIUM, MEDICAL IMAGE DISPLAY APPARATUS AND MEDICAL IMAGE DISPLAY SYSTEM

(71) Applicant: Konica Minolta, Inc., Tokyo (JP)

(72) Inventors: Takafumi NANJO, Tokyo (JP); Sumiya NAGATSUKA, Tokyo (JP); Ikki NAKAMURA, Tokyo (JP)

(21) Appl. No.: 17/852,872

(22) Filed: Jun. 29, 2022

(30) Foreign Application Priority Data
Jul. 26, 2021 (JP) 2021-121161

(51) Int. Cl. A61B 6/00; G06T 7/00; G06T 7/20

(52) U.S. Cl. CPC A61B 6/463; G06T 7/0012; G06T 7/20; G06T 2207/30061; G06T 2207/30101

(57) ABSTRACT

A non-transitory computer-readable storage medium stores a control program that causes a computer to perform first frame displaying. In the first frame displaying, the computer displays thumbnails of a series of frames next to each other on a display, the series of frames constituting a dynamic image of a subject or constituting a dynamic analysis image obtained by analyzing the dynamic image.

### CROSS-REFERENCE TO RELATED APPLICATIONS

The entire disclosure of Japanese Patent Application No. 2021-121161 filed on Jul. 26, 2021 is incorporated herein by reference in its entirety.

### TECHNICAL FIELD

The present invention relates to a storage medium, a medical image display apparatus, and a medical image display system.

### BACKGROUND

A picture archiving and communication system (hereinafter called PACS) receives image data of medical images (dynamic images, dynamic analysis images, still images, etc.) from modalities or image analysis apparatuses and stores the image data in a database.

An image interpretation terminal installed in a medical facility and connected to the PACS may display thumbnails of medical images stored in the PACS (for example, see JP2020-160369A). This allows doctors to select a medical image to be interpreted on the basis of the displayed thumbnails.

In response to receiving selection of a medical image to be interpreted, the image interpretation terminal obtains corresponding image data from the PACS and displays the medical image on the basis of the image data. The doctor then interprets the displayed medical image to diagnose the subject.

### SUMMARY

According to the known thumbnail display technique described in JP2020-160369A, one frame (still image) among frames constituting a dynamic image/dynamic analysis image is displayed as a thumbnail.

Thus, the known art, the user may not glance through thumbnails of a dynamic image.

For example, the thumbnail of the known art may not allow a doctor to recognize the feature of the interpretation-target image corresponding to the thumbnail; recognize motions of the subject analyzed to obtain the dynamic analysis image; or recognize whether the interpretation target image is a dynamic image or a still image.

The present invention has been conceived in view of the above issues. Objects of the present invention include improving browsability of thumbnails of medical images, such as a dynamic analysis image, as compared with the known art.

To achieve at least one of the above objects, according to an aspect of the present invention, there is provided a non-transitory computer-readable storage medium storing a control program that causes a computer to perform first frame displaying that is displaying thumbnails of a series of frames next to each other on a display, the series of frames constituting a dynamic image of a subject or constituting a dynamic analysis image obtained by analyzing the dynamic image.

According to another aspect of the present invention, there is provided a medical image display apparatus including a hardware processor configured to display thumbnails of a series of frames next to each other on a display, the series of frames constituting a dynamic image of a subject or constituting a dynamic analysis image obtained by analyzing a dynamic image.

According to another aspect of the present invention, there is provided a medical image display system including: an archive configured to archive frames constituting a dynamic image of a subject or frames constituting a dynamic analysis image obtained by analyzing the dynamic image; and a display apparatus configured to display thumbnails of a series of frames constituting the dynamic analysis image.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram showing an example of a medical image display system according to an embodiment of the present invention;

FIG. 2 is a block diagram of a medical image display apparatus included in the medical image display system according to this embodiment;

FIG. 3 is a sequence diagram showing an example of operation by the medical image display system;

FIG. 4 is an example of a display screen on the medical image display apparatus;

FIG. 5 is an example of a display screen on the medical image display apparatus;

FIG. 6 is an example of a display screen on the medical image display apparatus;

FIG. 7 is an example of a display screen on the medical image display apparatus;

FIG. 8 is an example of frames;

FIG. 9 is an example of a display screen on the medical image display apparatus; and

FIG. 10 is an example of a display screen on the medical image display apparatus.

### DESCRIPTION OF EMBODIMENTS

Hereinafter, an embodiment of the present invention is described with reference to the drawings. The scope of the present invention is not limited to the following embodiment or illustrated examples.

### 1. Overview of Medical Image Display System

An overview of a medical image display system (hereinafter, system 100) according to this embodiment is described.

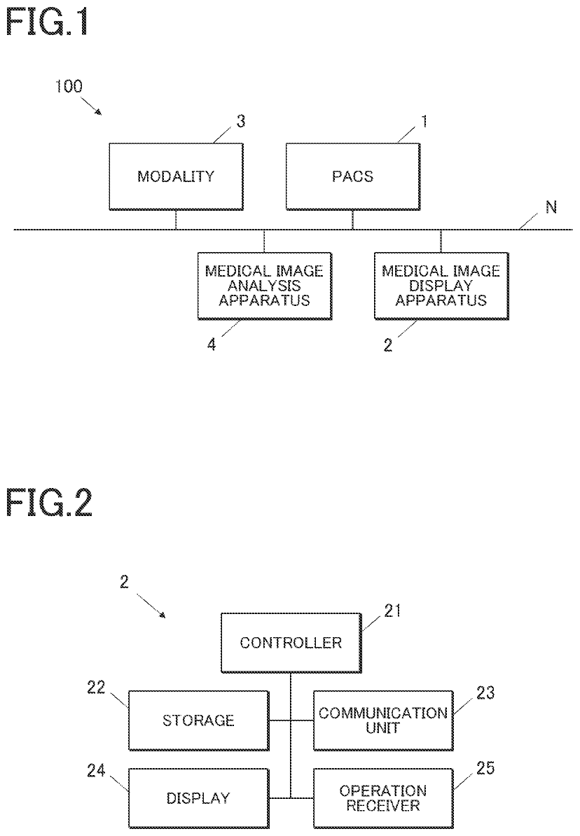

FIG. 1 is a block diagram of the system 100 as an example.

### [1-1. Schematic Configuration of Medical Image Display System]

The system 100 includes a picture archiving and communication system (hereinafter called PACS 1) and a medical image display apparatus (hereinafter called display apparatus 2) as shown in FIG. 1.

The system 100 according to this embodiment also includes a modality 3 and a medical image analysis apparatus (hereinafter called analysis apparatus 4).

The apparatuses 1 to 4 can communicate with each other over a communication network 5 (e.g., local area network (LAN), wide area network (WAN), the internet), for example.

The system 100 may be configured to communicate with not-illustrated hospital information system (HIS) and radiology information system (RIS).

### [Modality]

The modality 3 radiographs a subject (diagnosis target region of the subject) and generates digital data of medical images showing the subject (hereinafter called image data).

Examples of the modality 3 include a flat panel detector (FPD) device and a computed tomography (CT) device.

Examples of a medical image include a dynamic image, a dynamic analysis image obtained by analyzing the dynamic image (described in detail below), and a processed dynamic image (described in detail below), and a still image.

The dynamic image consists of multiple frames obtained through dynamic imaging of a motion of the subject.

The dynamic imaging is performed by repeating (i) repetitively irradiating the identical subject with pulsed radiation (or continuously irradiating the subject) from a radiation source and (ii) reading electric charges generated by imaging elements according to doses as signal values.

The dynamic image according to this embodiment is obtained by imaging a periodic motion of the subject.

Examples of the periodic motion of the subject include extraction and contraction of the lung field with breathing; pulsation of the heart; and blood flow through blood vessels of the lung field (the subject is the chest part). The examples of the periodic motion of the subject further include rotating, bending, and stretching of a joint (the imaging subject is the knee, elbow, or neck).

The modality 3 may include a not-illustrated console that is configured to set imaging conditions and control operations of components of the modality 3.

The modality 3 may be installed in an imaging room or configured to be movable.

### [Medical Image Analysis Apparatus]

The analysis apparatus 4 may be a PC, a dedicated device, or the like.

The analysis apparatus 4 analyzes a dynamic image obtained from the PACS 1 or the modality 3 and generates a dynamic analysis image.

The dynamic analysis image according to this embodiment consists of multiple frames obtained by analyzing the periodic motion of the subject.

Examples of the dynamic analysis image according to this embodiment include at least either a ventilation analysis image or a blood-flow analysis image.

The ventilation analysis image is the dynamic image on which the result of analyzing the expansion and contraction of the lung field is superposed.

The blood-flow analysis image is the dynamic image on which the result of analyzing the blood flow in blood vessels of the lung field is superposed.

The analysis apparatus 4 according to this embodiment also performs specific processing on the dynamic image obtained from the PACS 1 or the modality 3 to generate a processed dynamic image.

Examples of the processed dynamic image according to this embodiment include a frequency-emphasized image and a suppression image.

In the frequency-emphasized image, structures of the subject are shown more visibly.

In the suppression image, a specific structure (e.g., bone) is suppressed.

### [PACS]

The PACS 1 may be a PC, a dedicated device, or the like.

The PACS 1 at least stores multiple frames constituting the dynamic analysis image obtained from the analysis apparatus 4.

The PACS 1 according to this embodiment also stores frames constituting the dynamic image obtained from the modality 3; frames constituting the processed dynamic image; and still images.

The PACS 1 according to this embodiment stores multiple pieces of image data together with corresponding examination information in a database.

The PACS 1 according to this embodiment thus serves as an archive.

On the basis of stored medical image data (dynamic image, dynamic analysis image, processed dynamic image, still image, etc.) the PACS 1 according to this embodiment generates thumbnails of the corresponding medical images.

The thumbnails generated by the PACS 1 are also included in the medical images in this embodiment.

Thumbnails of the dynamic image, dynamic analysis image, and processed dynamic image consist of multiple frames, as with the original dynamic image, dynamic analysis image, and processed dynamic image.

### [Image Display Apparatus]

The display apparatus 2 may be a PC, a dedicated device, or the like.

The display apparatus 2 at least displays multiple frames constituting the dynamic analysis image obtained from the PACS 1.

The display apparatus 2 according to this embodiment also displays frames constituting the dynamic image, still images, and other images.

The display apparatus 2 may be configured to display medical images obtained directly (i.e., not via the PACS 1) from the modality 3 or the analysis apparatus 4.

The display apparatus 2 is described later in detail.

[1-2. Diagnosis Flow with Image Display System]

With the system 100 configured as described above, diagnosis is done as follows.

The user firstly uses the modality 3 to image the subject. The modality 3 generates image data of a medical image showing the subject (dynamic image or still image).

The analysis apparatus 4 obtains the image data of the dynamic image from the modality 3 or from the PACS 1, analyzes the dynamic image, and generates image data of a dynamic analysis image.

The PACS 1 obtains image data from the modality 3 or from the analysis apparatus 4 and stores the obtained image data.

The display apparatus 2 obtains image data from the PACS 1, the modality 3, or the analysis apparatus 4 and displays the medical image on the basis of the obtained image data.

The doctor interprets the medical image displayed on the display apparatus 2 and diagnoses the subject.

### [1-3. Modification of Image Display System]

The above-described system 100 includes the PACS 1, the display apparatus 2, the modality 3, and the analysis apparatus 4. However, when at least one of the PACS 1, the modality 3, and the analysis apparatus 4 has a display, the apparatus 1/3/4 having the display may perform at least partial functions of the display apparatus 2.

In the case, the system 100 may not include the display apparatus 2.

Further, the PACS 1, the display apparatus 2, or the modality 3 may perform at least partial functions of the analysis apparatus 4.

In the case, the system 100 may not include the analysis apparatus 4.

Further, the display apparatus 2, the modality 3, or the analysis apparatus 4 may perform at least partial functions of the PACS 1.

In the case, the system 100 may not include the PACS 1.

In the above-described system 100, the PACS 1 generates image data of thumbnails. However, the display apparatus 2 may generate the image data of thumbnails.

### 2. Detailed Configuration of Image Display Apparatus

Next, the display apparatus 2 of the system 100 is described in detail.

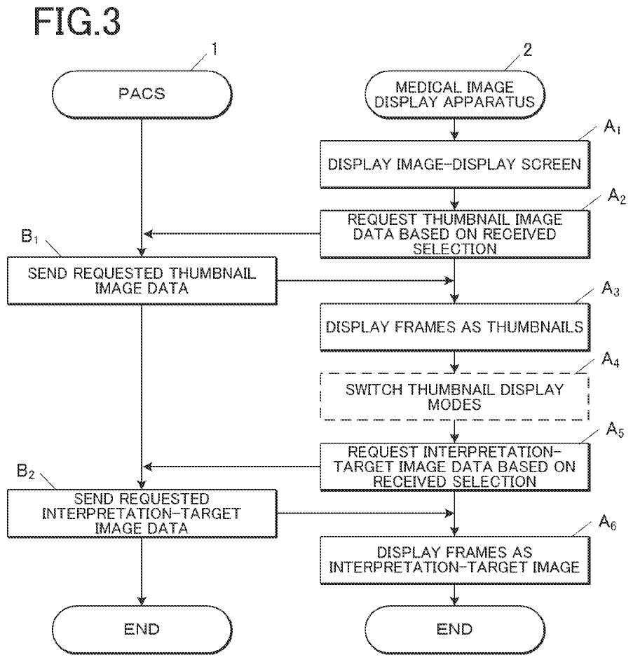

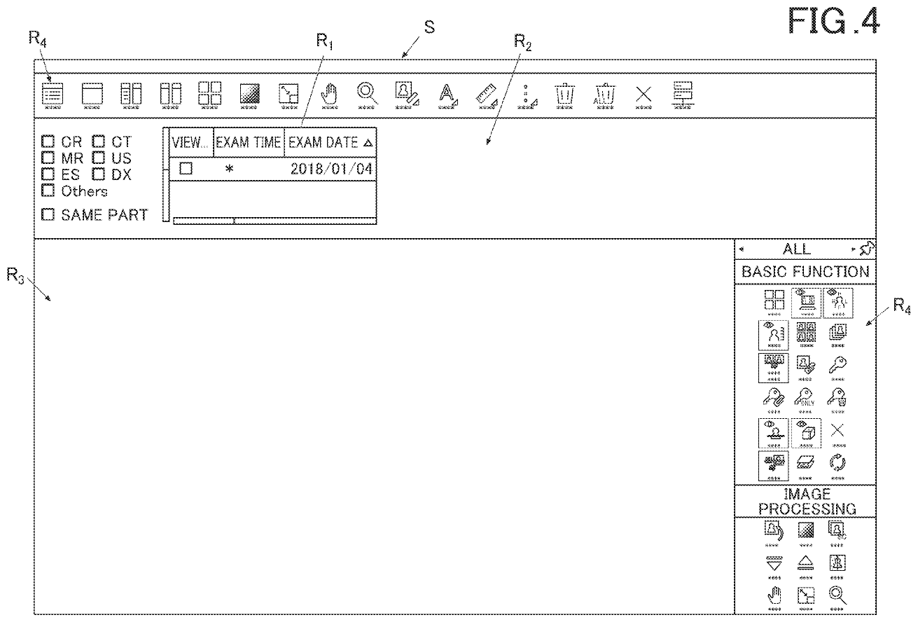

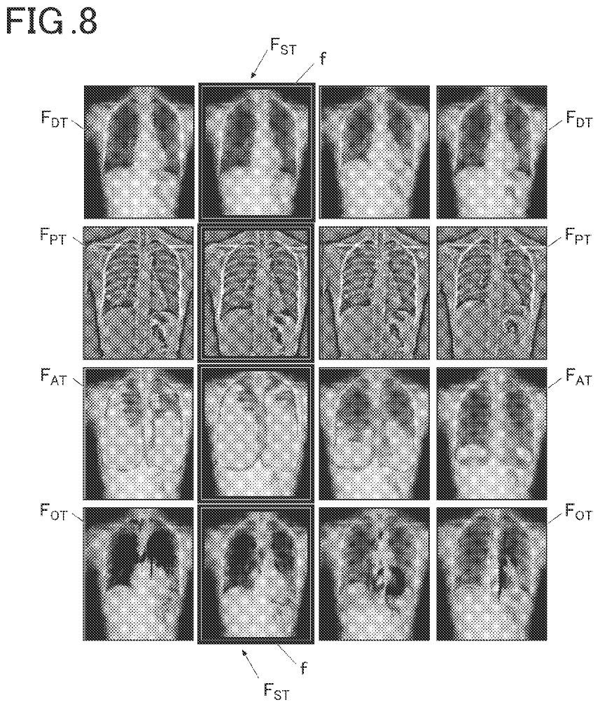

FIG. 2 shows a block diagram of the display apparatus 2. FIG. 3 shows a sequence diagram showing an example of operation by the system 100. FIGS. 4-7, 9, 10 show examples of screens displayed by the display apparatus 2. FIG. 8 shows an example of frames.

### [2-1. Configuration of Image Display Apparatus]

As exemplified in FIG. 2, the display apparatus 2 includes a controller 21 (hardware processor), a storage 22, a communication unit 23, a display 24, and an operation receiver 25.

These components 21-25 are electrically connected via a bus, for example.

The controller 21 includes a central processing unit (CPU) and a random access memory (RAM).

The CPU of the controller 21 reads various programs stored in the storage 22, loads the programs into the RAM, and performs various processes in accordance with the loaded programs. The CPU of the controller 21 thus centrally controls operations of the components of the display apparatus 2.

The storage 22 consists of a nonvolatile memory and/or a hard disk, for example.

The storage 22 stores various programs including the control program to be executed by the controller 21 and parameters necessary for executing the programs.

The storage 22 may be configured to store image data of medical images.

The communication unit 23 includes a communication module.

The communication unit 23 sends and receives various signals and data to and from other apparatuses (e.g., PACS 1, modality 3, and analysis apparatus 4) wirelessly or through wires over the communication network 5.

The display 24 consists of a liquid crystal display (LCD) or cathode ray tube (CRT), for example.

The display 24 displays medical images and so forth on the basis of image signals received from the controller 21.

The operation receiver 25 includes a keyboard with cursor keys, number keys, and various function keys, a pointing device such as a mouse, and a touchscreen layered on the surface of the display 24.

The operation receiver 25 outputs control signals corresponding to the user's operation to the controller 21.

The display apparatus 2 may not include the display 24 and the operation receiver 25. For example, the display apparatus 2 may receive control signals from other devices (a dedicated input device, the PACS 1, the analysis apparatus 4, etc.) and output image signals to other devices (a dedicated monitor, the PACS 1, the analysis apparatus 4, etc.) via the communication unit 23, for example.

### [2-2. Operation of Medical Image Display Apparatus]

The controller 21 of the display apparatus 2 configured as described above performs the following operation.

For example, when a predetermined condition is met, the controller 21 performs a medical image display process on the basis of the control program stored in the storage 22.

Examples of the predetermined condition include (i) the display apparatus is turned on; (ii) image data is obtained from other devices; (iii) control signals are received from other devices; and (iv) a predetermined operation is made with the operation receiver 25.

The medical image display process includes a first frame-displaying step A3 as shown in FIG. 3.

The medical image display process in this embodiment further includes the screen displaying step A1, first request step A2, switch step A4, second request step A5, and second frame-displaying step A6.

### [Display Image-Display Screen]

In the medical image display process in this embodiment, the controller 21 firstly performs the screen displaying step A1.

In the screen displaying step A1, the controller 21 causes the display 24 to display the image-display screen S.

The image-display screen S in this embodiment includes an examination-information display region R1, a thumbnail display region R2, an interpretation-target-image display region R3, and an operation-button display region R4, as exemplified in FIG. 4.

The screen displaying step A1 is not required when the display 24 displays medical images only and does not display others (examination information, operation buttons, and so forth).

### [Request and Obtain Image Data of Thumbnails]

After displaying the image-display screen S, when a specific selection operation is received from the user, the controller 21 executes the first request step A2 as shown in FIG. 3.

The selection operation is a click or touch on a piece of examination information among multiple pieces of examination information displayed on the examination-information display region R1, for example.

In the first request step A2, the controller 21 requests image data of thumbnails of the medical image corresponding to the received selection operation from the PACS 1 via the communication unit 23.

When receiving the request, the PACS 1 sends the requested image data of thumbnails to the display apparatus 2 (Step B1). The controller 21 thus obtains the image data.

In the first request step A2, the controller 21 may request and obtain image data from a device other than the PACS 1.

The PACS 1 may be configured to receive the selection operation. In the case, this first request step A2 is not required.

### [Display Thumbnails]

After obtaining the image data of thumbnails, the controller 21 executes the first frame-displaying step A3 (first frame displaying).

In the first frame-displaying step A3, the controller 21 displays the thumbnails of the medical image next to each other on the display 24, on the basis of the image data obtained from the PACS 1.

When the medical image obtained in the first request step A2 is a dynamic analysis image, the controller 21 displays the thumbnails of frames F_{AT} next to each other on the display 24. Herein, the frames F_{AT} are a series of frames constituting the dynamic analysis image.

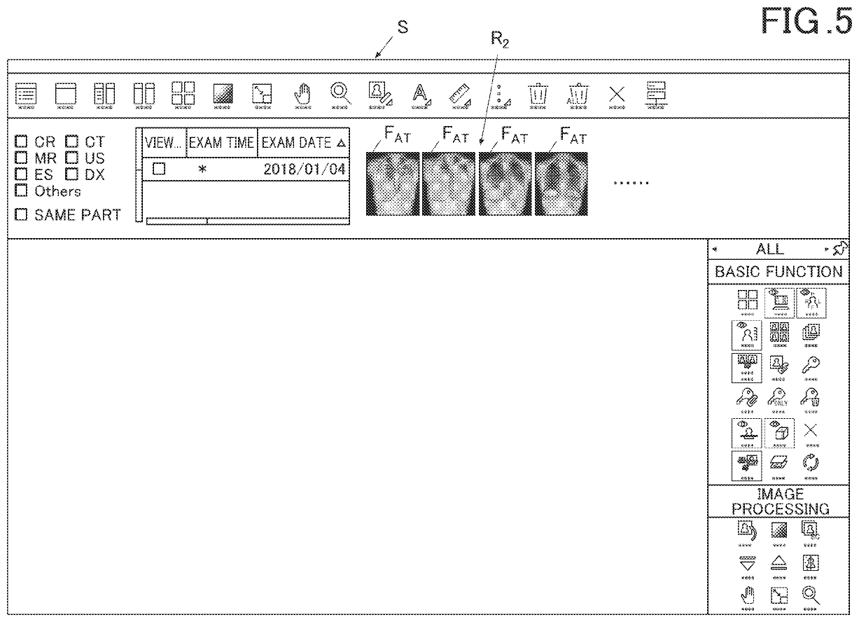

In the first frame-displaying step S3 according to this embodiment, the controller 21 displays the frames F_{AT} in the thumbnail display region R2, as shown in FIG. 5.

In the first frame-displaying step S3 according to this embodiment, the controller 21 displays the frames F_{AT} in chronological order or in the order of frame numbers (the order of obtaining the frames).

The frames F_{AT} may be displayed next to each other in a first display mode or a second display mode.

In the first display mode, the frames F_{AT} are displayed side by side in a spatial direction at one time on the same display screen, as shown in FIG. 5 (i.e., the frames F_{AT} are tiled).

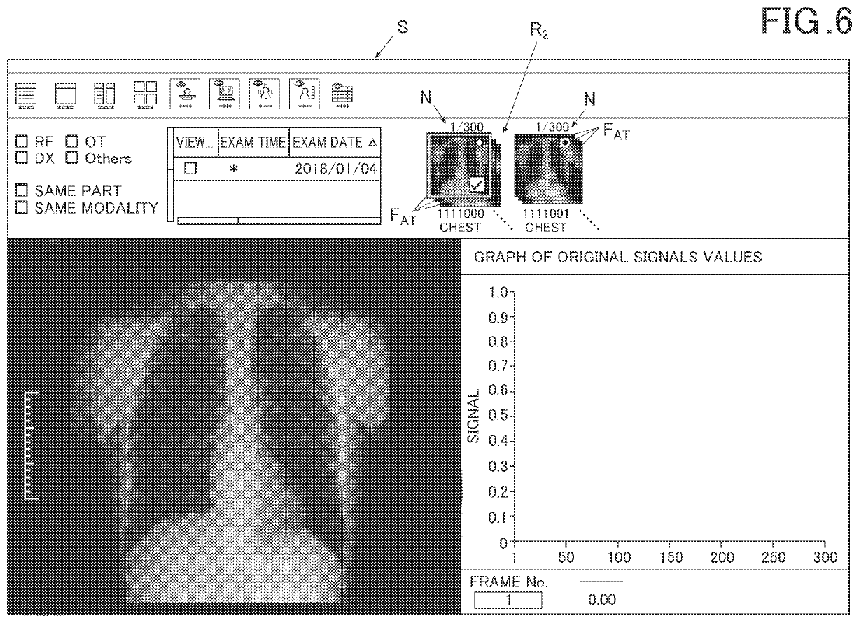

In the second display mode, the frames F_{AT} are arranged in chronological order in a specific region of the display screen and sequentially switched (i.e., the frames F_{AT} are played as a video), as exemplified in FIG. 6.

In the second display mode, the controller 21 may play the movie consisting of thumbnails only once or may play the movie repetitively.

By viewing thumbnails of frames F_{AT} displayed in the first display mode or second display mode, the user can easily recognize the feature of the dynamic image (interpretation-target image) corresponding to the thumbnails; recognize motions of the subject that have been analyzed to obtain the dynamic analysis image; and recognize whether the interpretation target image corresponding to the thumbnails is a dynamic image or a still image.

In particular, when the frames F_{AT} show the periodic motion of the subject, the user can easily recognize phases of the frames F_{AT} by simply viewing thumbnails of the frames F_{AT} displayed in the first display mode or the second display mode.

In the first frame-displaying step S3, the controller 21 may display as many thumbnail frames F_{AT} as the frames F_{AT} constituting the corresponding interpretation-target image. Alternatively, the controller 21 may display less thumbnail frames F_{AT} than the frames F_{AT} constituting the interpretation-target image (i.e., thin out the thumbnail frames F_{AT}).

In displaying the thumbnail frames F_{AT} in the thumbnail display region R2 in the second display mode, the frame rate of the thumbnail frames F_{AT} may be the same or different from the frame rate of the corresponding interpretation target image.

In displaying the thumbnail frames F_{AT} in the thumbnail display region R2 in the second display mode, the frame number N of a frame F_{AT} being displayed may be shown near the frame F_{AT}, as shown in FIG. 6.

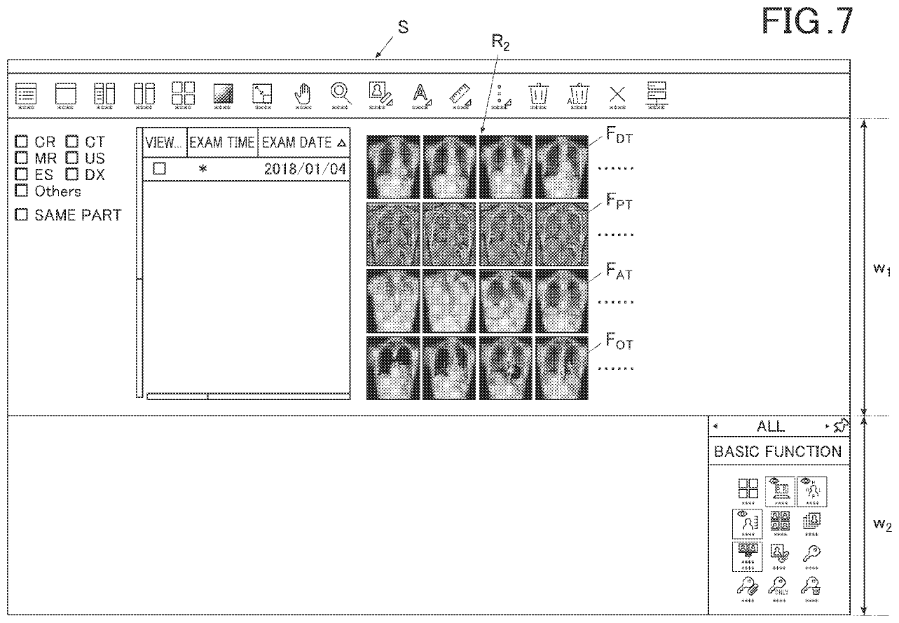

In the first frame-displaying step S3 according to this embodiment, the controller 21 may display multiple sets of thumbnails simultaneously, as exemplified in FIG. 7.

For example, the controller 21 may display thumbnail frames F_{AT} in one series constituting the dynamic analysis image and thumbnail frames F_{DT} in one series constituting the dynamic image, which is the original of the dynamic analysis image, next to each other on the display 24.

The controller 21 may also display thumbnail frames F_{AT} in one series constituting the dynamic analysis image and thumbnail frames F_{PT} in one series constituting the processed dynamic image, which is obtained by performing predetermined image processing on the dynamic image, next to each other on the display 24.

When multiple types of dynamic analysis images are present, the controller 21 may display thumbnail frames F_{AT} in one series constituting the dynamic analysis image and thumbnail frames F_{OT} in one series constituting the other dynamic analysis image next to each other on the display 24.

The controller 21 may display thumbnail frames F_{AT} in one series constituting the dynamic analysis image and still images next to each other on the display 24.

When a predetermined condition is met, in this first frame-displaying step S3, the controller 21 may display only the thumbnail frames F_{DT} in one series constituting the dynamic image; only the thumbnail frames F_{PT} in one series constituting the processed dynamic analysis image; or a still image(s). Examples of the predetermined condition include (i) only the image data of the dynamic image, the processed dynamic image, or the still image is obtained; and (ii) the user has selected only the dynamic image, the processed dynamic image, or the still image.

The dynamic analysis image, the dynamic image, and the processed dynamic image may be divided into clips (e.g., by every n cycles) on the basis of the periodic motion of the subject. In the case, the controller 21 may display the thumbnail frames F_{AT}, frames F_{DT}, frames F_{OT}, and frames F_{PT} by clips as the frames showing the respective clips.

When multiple sets of thumbnails are present (i.e., thumbnails of the dynamic image, dynamic analysis image, processed dynamic image, and/or still image are present), the controller 21 may display only part of the multiple sets of thumbnails in the first frame-displaying step A3.

In the case, the controller 21 may select the set(s) of thumbnails to be displayed on the basis of specific manual selection by the user. Alternatively, the controller 21 may automatically select the thumbnails to be displayed.

When automatically selecting the thumbnails to be displayed, the controller 21 may select the thumbnails on the basis of the clinical department (the department to which the doctor belongs or the department in which the display apparatus 2 is installed) or the information attached to the dynamic image (e g, examination information, series information).

When the dynamic analysis image and another medical image are displayed in the first display mode, multiple sets of thumbnails are displayed in a vertical arrangement.

In such a case, in the first frame-displaying step S3 in this embodiment, the controller 21 widens/narrows the vertical width W1 of the thumbnail display region R2 and narrows/widens the vertical width W2 of the interpretation-target-image display region R3, depending on the number of sets of thumbnails displayed.

Further, in the first frame-displaying step S3 in this embodiment, the controller 21 displays a specific frame F_{ST} among the frames F_{AT}, F_{DT}, F_{OT}, F_{PT}, such that the specific frame F_{ST} is distinguishable from the other frames F_{AT}, F_{DT}, F_{OT}, F_{PT}.

The specific frame F_{ST} may be a key frame, a first (top) frame, a middle frame, a last frame, or a frame of a specific frame number, for example.

The key frame serves as a key in making a diagnosis. Examples of the key frame include a frame showing a lesion most clearly and a frame showing the largest/smallest motion (e.g., expansion of the lung field, blood flow of a specific blood vessel).

In the first frame-displaying step A3, the controller 21 may select the specific frame F_{ST} automatically or on the basis of manual selection by the user.

The controller 21 may automatically select the specific frame F_{ST} by referring to predetermined selection criteria or by inputting new frames F_{AT}, F_{DT}, F_{OT}, F_{PT} to a trained model. The trained model may be trained through machine learning on the basis of multiple frames as inputs and specific frames as outputs.

To display the specific frame F_{ST} to be distinguishable from the other frames F_{AT}, F_{DT}, F_{OT}, F_{PT}, the specific frame F_{ST} may be displayed with a figure that is not shown for the other frames F_{AT}, F_{DT}, F_{OT}, F_{PT} (e.g., the specific frame F_{ST} enclosed in a framework f as shown in FIG. 8); the specific frame F_{ST} may be displayed with an annotation that is not shown for the other frames F_{AT}, F_{DT}, F_{OT}, F_{PT}; the specific frame F_{ST} may be colored differently from the other frames F_{AT}, F_{DT}, F_{OT}, F_{PT}; the specific frame F_{ST} may be relatively larger than the other frames F_{AT}, F_{DT}, F_{OT}, F_{PT}; or the specific frame F_{ST} may have a better image quality than the other frames F_{AT}, F_{DT}, F_{OT}, F_{PT}, for example.

When a set of thumbnails of the other medical image is displayed together with the set of thumbnails of the dynamic analysis image in the first frame-displaying step S3, the controller 21 may determine the specific frame F_{ST} for each individual set of thumbnails.

Further, assume that at least a set of thumbnails of the dynamic image, the other dynamic analysis image, or the processed dynamic image is displayed together with the set of thumbnails of the dynamic analysis image in the first frame-displaying step A3; and that the specific frame F_{ST} of a specific frame number is determined for any set of thumbnail frames F_{AT}, F_{DT}, F_{OT}, or F_{PT}. In the case, the controller 21 may automatically determine the frame(s) of the specific frame number in the remaining set(s) of thumbnail frames F_{AT}, F_{DT}, F_{OT}, or F_{PT} to be the specific frame(s) F_{ST}.

### [Switch Display Modes]

In the medical image display process according to this embodiment, when the user performs switching operation with the operation receiver 25 while the thumbnails are displayed, the controller 21 executes the switch step A4 shown in FIG. 3.

The switching operation is a click or touch on a display-mode selection button shown in the operation-button display region R4, for example.

In the switch step A4, the controller 21 switches the first/second display mode in which the frames F_{AT}, F_{DT}, F_{OT}, or F_{PT} are currently displayed to the other display mode (second/first display mode).

Thus, the user can check the thumbnails in multiple ways to recognize the medical image corresponding to the thumbnails.

In the switch step A4, the controller 21 may switch the display modes for all sets of thumbnails at one time or may switch the display modes for the respective sets of thumbnails.

In the switch step A4, the controller 21 may automatically switch the display modes when a predetermined time elapses from the start of the first frame-displaying step S3.

### [Request and Obtain Image Data of Interpretation-Target Image]

In the medical image display process, when any of thumbnails displayed on the display 24 is clicked or touched, the controller 21 executes the second request step A5 shown in FIG. 3.

In the second request step A5, the controller 21 requests image data of the interpretation-target image corresponding to the selected thumbnails from the PACS 1 via the communication unit 23.

When receiving the request, the PACS 1 sends the requested image data of the interpretation-target data to the display apparatus 2 (Step B2). The controller 21 thus obtains the image data.

In the second request step A5, the controller 21 may request and obtain image data from a device other than the PACS 1.

Further, the PACS 1 may be configured to receive the selection operation. In the case, this second request step A5 is not required.

### [Display Interpretation-Target Image]

After obtaining the image data of the interpretation-target image, the controller 21 executes the second frame-displaying step A6 shown in FIG. 3.

In the second frame-displaying step A6, the controller 21 displays the interpretation-target image on the display 24 on the basis of the image data obtained from the PACS 1.

When the medical image obtained in the second request step A5 is a dynamic analysis image, the controller 21 displays frames F_{AI} in one series constituting the dynamic analysis image next to each other on the display 24.

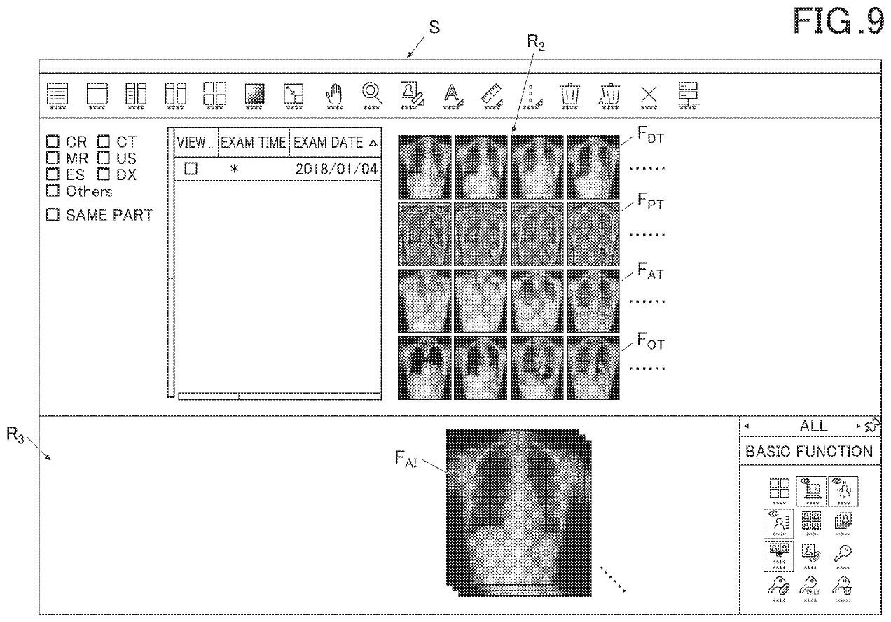

In the second frame-displaying step A6, the controller 21 displays the interpretation-target image in the interpretation-target-image display region R3 of the image-display screen S, as shown in FIG. 9, for example.

The way of displaying the interpretation-target image is basically the same as the way of displaying thumbnails described above, except the region in which the frames are displayed, the size of the displayed images, and the image quality, for example.

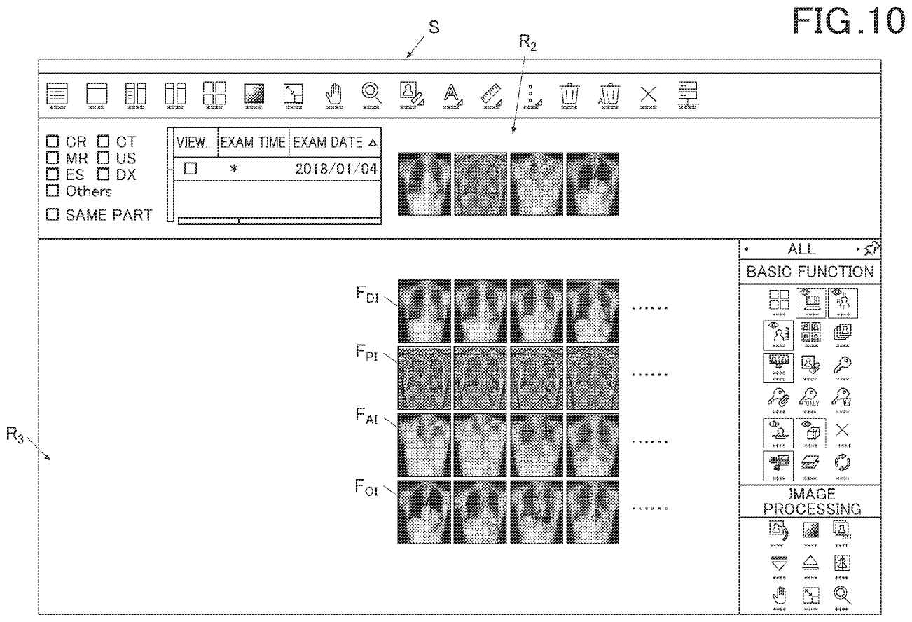

That is, in the second frame-displaying step A6 in this embodiment, the controller 21 may display at least one of the following on the display 24 as the interpretation-target image: frames F_{AI} in one series constituting the dynamic analysis image; frames F_{DI} in one series constituting the dynamic image; frames F_{DI} in one series constituting the other dynamic analysis image; and a still image(s), as shown in FIG. 10.

When the multiple sets of frames F_{AI}, F_{DI}, F_{OI}, and/or F_{PI} are displayed next to each other, the frames may be displayed in the first display mode as shown in FIG. 10 or the second display mode as shown in FIG. 9.

In the second frame-displaying step A6, the controller 21 may display multiple sets of frames F_{AI}, F_{DI}, F_{OI}, and/or F_{PI} on the basis of clips.

Further, the controller 21 may display a specific frame to be distinguishable from the other frames F_{AI}, F_{DI}, F_{OI}, and/or F_{PI}.

Further, the controller 21 may switch the display modes from the first/second display mode in which the frames F_{AI}, F_{DI}, F_{DI}, or F_{PI} are currently displayed to the other display mode.

When at least one of the set of thumbnails of the dynamic analysis image, the set of thumbnails of the dynamic image, and the set of thumbnails of the processed dynamic image is displayed in the first display mode, the user is allowed to select one thumbnail frame from among the thumbnail frames F_{AT}, F_{DT}, F_{OT}, F_{PT} in the second request step A5.

Therefore, in the second frame-displaying step A6, the controller 21 may start displaying the interpretation-target image from the frame F_{AI}, F_{DI}, F_{OI}, F_{PI} that correspond to the selected frame F_{AT}. Alternatively, the controller 21 may display only the frames F_{AI}, F_{DI}, F_{OI}, F_{PI} that constitute the clip including the selected frames F_{AT}, F_{DT}, F_{OT}, F_{PT}.

As described above, in this embodiment, the vertical width W2 of the interpretation-target-image display region R3 is narrowed when the number of sets of thumbnails displayed in the first display mode in the thumbnail display region R2 is increased.

When the number of sets of thumbnails displayed in the first display mode in the thumbnail display region R2 is equal to or greater than a predetermined number, in the second frame-displaying step A6, the controller 21 may reduce the size of the interpretation-target image according to the vertical width W2. Alternatively, the controller 21 may move the interpretation-target image downward (the bottom of the interpretation-target image is out of screen) or may not display the interpretation-target image.

In the case, the controller 21 may change the layout of the image-display screen S. For example, the thumbnail display region may be at one side of the display in the horizontal direction, and the interpretation-target-image region may be at the other side of the display.

### 3. Advantageous Effects

As described above, according to this embodiment, the control program causes the display apparatus 2 (computer) to perform the first frame-displaying step S3 (first frame displaying) that is displaying thumbnails of frames F_{DT} in one series constituting the dynamic image or thumbnails of frames F_{AT} in one series constituting the dynamic analysis image next to each other on the display 24.

The control program, the display apparatus 2 storing the control program in the storage 22, and the system 100 including the display apparatus 2 can improve viewability of thumbnails of medical images including dynamic analysis images.

### 4. Modification

Naturally, the above embodiment does not limit the present invention and can be appropriately modified without departing from the scope of the present invention.

In the above embodiment, the display apparatus 2 is mainly configured to display thumbnails of frames constituting a medical image of any kind next to each other. However, the display apparatus 2 may be mainly configured to display frames constituting a medical image in the first display mode no matter whether the frames are a set of thumbnails or an interpretation-target image. The display apparatus 2 may also be configured to display frames constituting a medical image in the first display mode as an interpretation-target image.

In the above embodiment, the display apparatus 2 displays thumbnails of frames in one series constituting a dynamic image/dynamic analysis image next to each other on the display. The display apparatus 2 may display each frame in multiple series as one thumbnail.

Further, in the above description, a hard disk and a semiconductor nonvolatile memory are disclosed as computer readable media for storing the control program of the present invention. However, these examples do not limit the present invention. As other computer readable media, a portable storage medium, such as a CD-ROM, can be used.

A carrier wave is also applicable as a medium for providing the control program data of the present invention via a communication line.

What is claimed is:

1. A non-transitory computer-readable storage medium storing a control program that causes a computer to perform first frame displaying that is displaying thumbnails of a series of frames next to each other on a display, the series of frames constituting a dynamic image of a subject or constituting a dynamic analysis image obtained by analyzing the dynamic image.

2. The storage medium according to claim 1, wherein the dynamic analysis image is obtained by analyzing a periodic motion of the subject.

3. The storage medium according to claim 2, wherein
the subject is a chest part, and
the dynamic analysis image includes a ventilation analysis image obtained by analyzing expansion and contraction of a lung field and/or a blood-flow analysis image obtained by analyzing a blood flow in blood vessels of the lung field.

4. The storage medium according to claim 1, wherein
the program further causes the computer to perform second frame displaying that is displaying the frames constituting the dynamic analysis image next to each other as an interpretation-target image on the display.

5. The storage medium according to claim 1, wherein
the program causes the computer to perform the first frame displaying such that a specific frame among the frames is distinguishable from the frames other than the specific frame.

6. The storage medium according to claim 1, wherein
the program causes the computer to display the frames next to each other in a first display mode and a second display mode,
in the first display mode, the frames are displayed side by side simultaneously on an identical display screen, and
in the second display mode, the frames are displayed by being sequentially switched from one to another in a specific region of the display screen.

7. The storage medium according to claim 6, wherein
the program further causes the computer to switch display modes from the first or second display mode in which the frames are currently displayed to the other display mode.

8. The storage medium according to claim 1, wherein
in the first frame displaying, the program causes the computer to display the frames in chronological order or in order of frame numbers.

9. A medical image display apparatus comprising a hardware processor configured to display thumbnails of a series of frames next to each other on a display, the series of frames constituting a dynamic image of a subject or constituting a dynamic analysis image obtained by analyzing a dynamic image.

10. A medical image display system comprising:
an archive configured to archive frames constituting a dynamic image of a subject or frames constituting a dynamic analysis image obtained by analyzing the dynamic image; and
a display apparatus configured to display thumbnails of a series of frames constituting the dynamic analysis image.

11. The medical image display system according to claim 10, further comprising an analysis apparatus configured to analyze the dynamic image and generate the dynamic analysis image.

12. The medical image display system according to claim 11, further comprising a modality configured to dynamically image a motion of the subject and generate the dynamic image.
